(19) United States
(12) Plant Patent Application Publication
(10) Pub. No.: US 2016/0073562 P1
(43) Pub. Date: Mar. 10, 2016

(54) "Variety of cherry tree named 'Areko"

(50) ;

(71) Applicant: Julius Kühn-Institut (JKI), Federal Research Centre for Cultivated Plants, Quedlinburg (DE)

(72) Inventor: Mirko Schuster, Dresden (DE)

(21) Appl. No.: 14/121,482

(22) Filed: Sep. 10, 2014

(51) Int. Cl. A01H 5/00

(52) U.S. Cl. PLT/181

(57) ABSTRACT

A new and distinct cherry tree that produces sweet cherry fruits with very good eating qualities.

### BOTANICAL CLASSIFICATION

Prunus avium

### VARIETAL DENOMINATION

‘Areko’

### BACKGROUND OF THE INVENTION

The present invention comprises a new and distinct cultivar of Prunus avium known by the varietal name ‘Areko’ (also internally referred to as “Hamid”). The new variety was discovered in Dresden, Germany after many years of controlled breeding. The new variety is the result of a planned breeding program between cherry trees named ‘Kordia’ (female parent, unpatented) and ‘Regina’ (male parent, unpatented). The new variety is similar to ‘Kordia’ in ripening time and ‘Regina’ in fruit size. The new variety differs from its parental varieties in having a pointed fruit pistil end and an overall cordate fruit shape. When further compared to ‘Kordia’, the new variety is similar in fruit color, but has a heavier fruit weight and lighter red color of its fruit flesh than ‘Kordia’. The purpose of breeding the new variety was to provide cherries with improved fruit quality. The new variety was first asexually reproduced via grafting in Ahrensburg, Germany. The new variety has been trial and field tested and has been found to retain its distinctive characteristics and remain true to type through successive propagations. The following characteristics distinguish the new variety from other unpatented cherry tree varieties known to the breeder:

1. ‘Lapins’—Blooming time (early);

2. ‘Krupnoplodnaja’—Fruit shape (reniform);

3. ‘Burlat’—Ripening time (early);

4. ‘Staccato’—Ripening time (very late);

5. ‘Büttners Rote Knorpelkirsche’—Fruit color; and

6. ‘Vanda’—Fruit shape (reniform).

### DESCRIPTION OF THE DRAWINGS

The accompanying photographic drawings illustrate the new variety, with the color being as nearly true as is possible with color illustrations of this type:

FIG. 1 is a close-up photograph of the fruit of ‘Areko’;

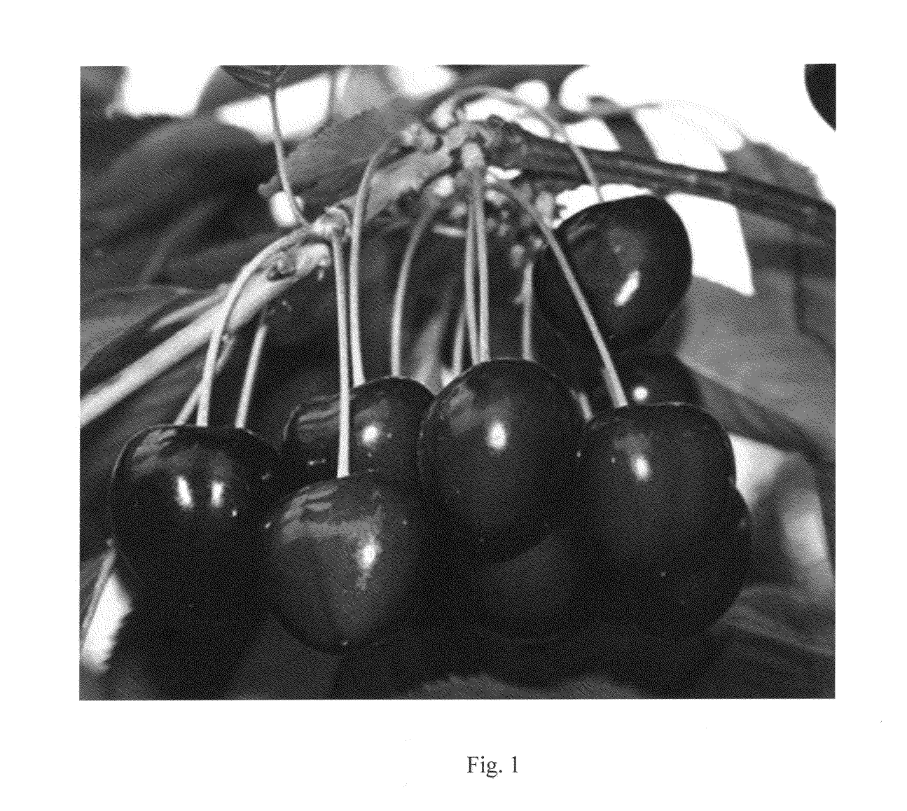

FIG. 2 is a further close-up photograph of the fruit of ‘Areko’;

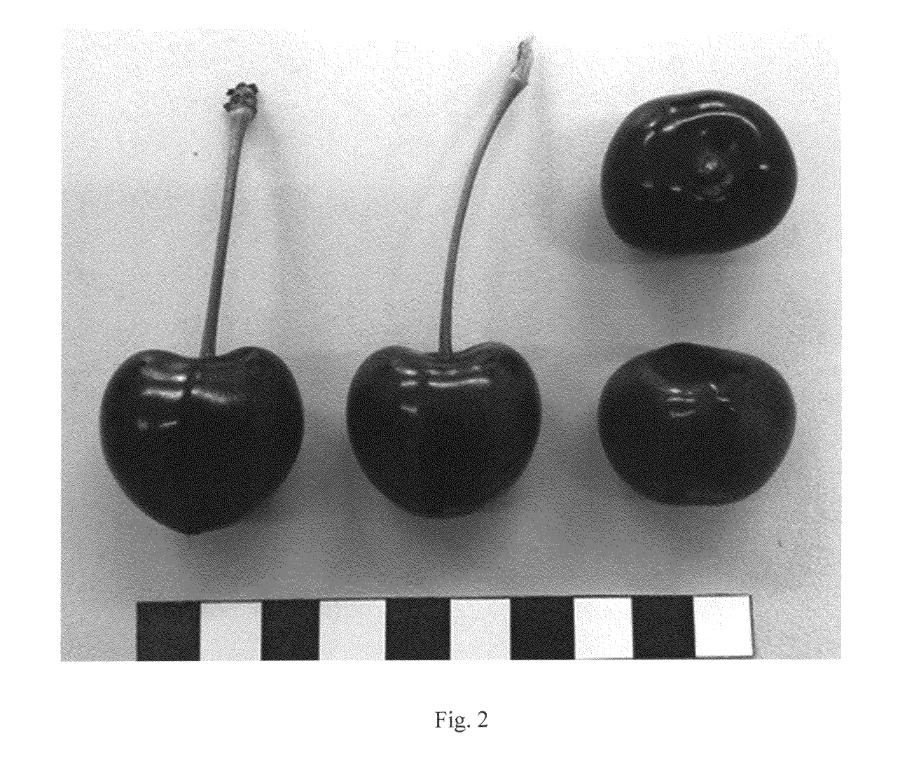

FIG. 3 is a comparative photograph of the fruit of ‘Areko’ (“Hamid”);

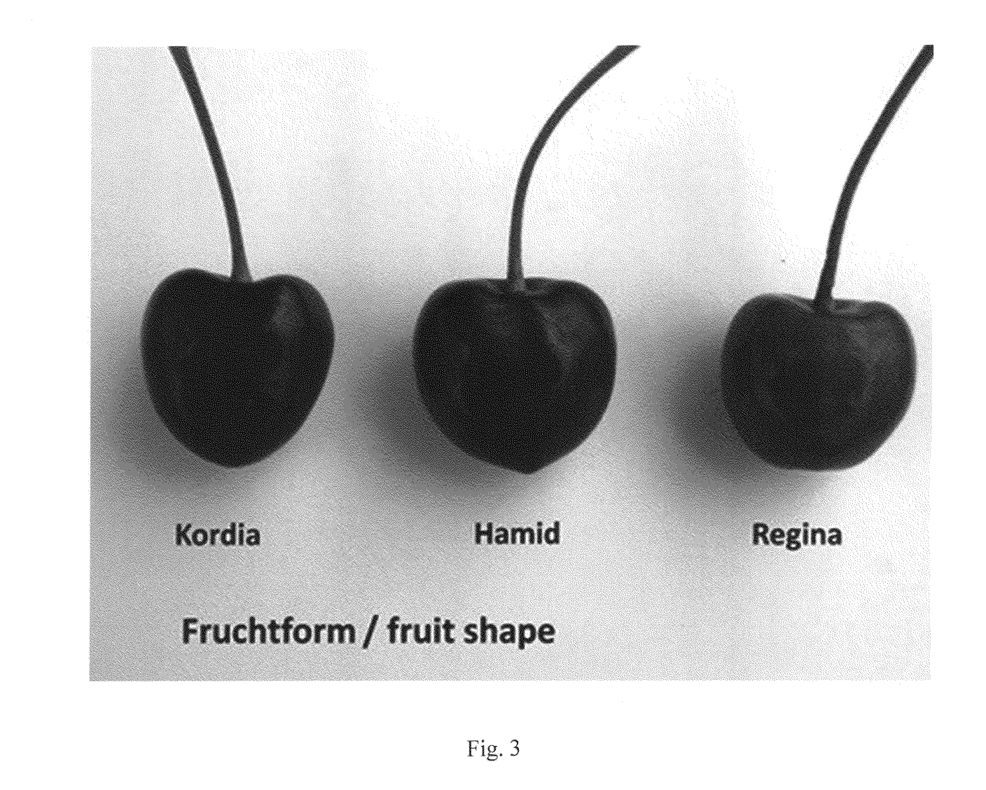

FIG. 4 is a photograph of the flowers of ‘Areko’;

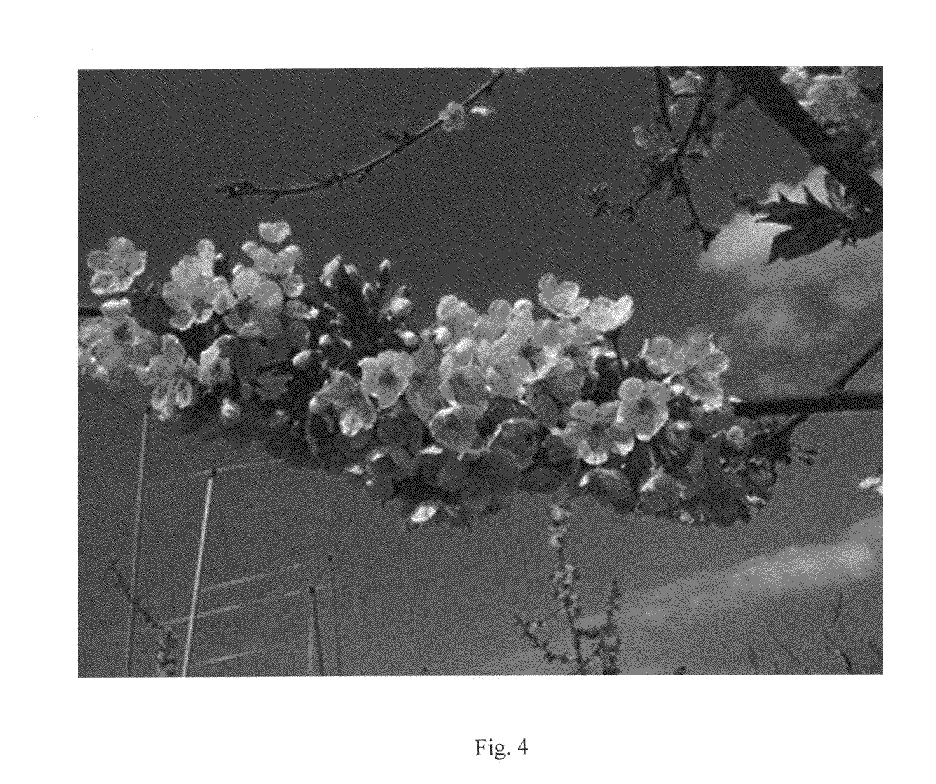

FIG. 5 is a close-up photograph of the leaves of ‘Areko’; and

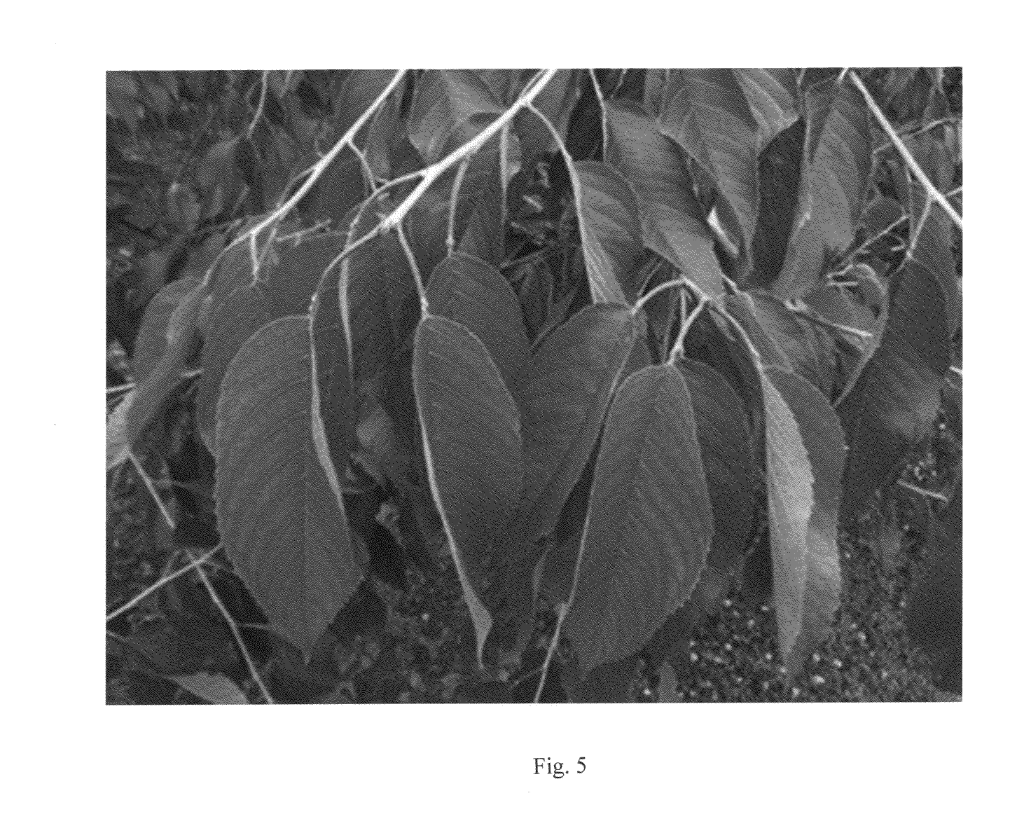

FIG. 6 is a photograph of an entire tree of ‘Areko’.

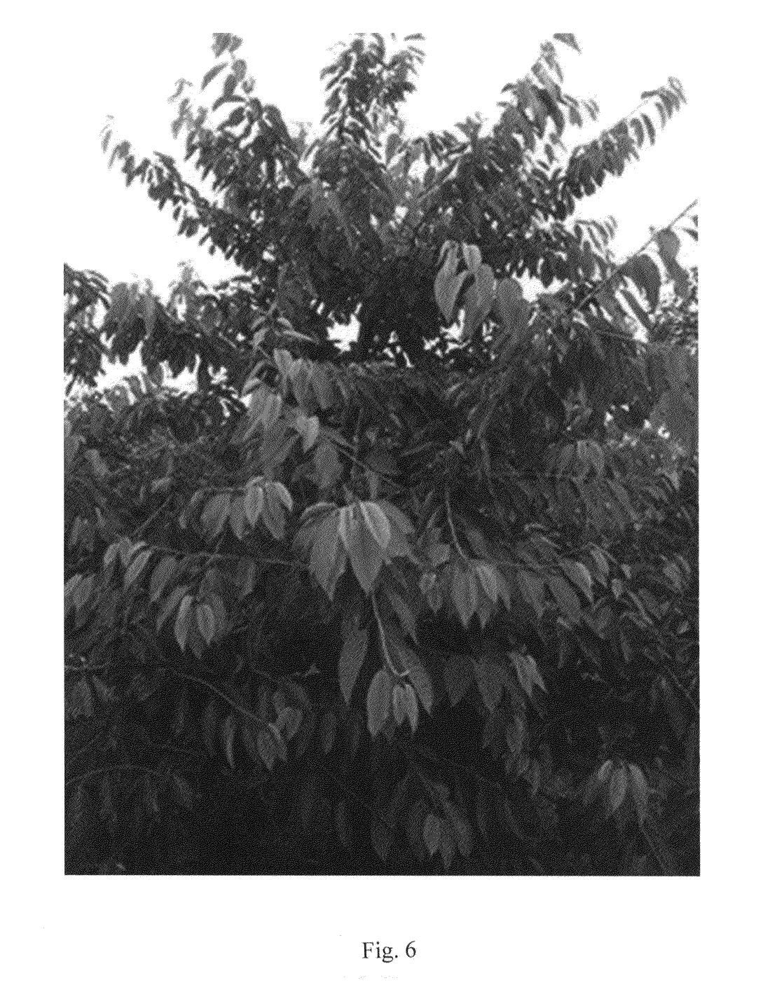

### DETAILED BOTANICAL DESCRIPTION

The following detailed description sets forth the characteristics of the new cultivar. The new variety was grown under standard cherry tree growing conditions using a central leader (spindle) training system in Dresden, Germany. Fungicide and insecticide treatments were provided according to general recommendations. The color readings were taken in natural daylight, and the plant was 7 years old when described. The color standard is the #6 color chip of the Centre Technique Interprofessionnel des Fruits (CTIFL) color chart.
Tree:
   Height.—2.5-3.0 m.
   Spread.—2.5-4.5 m.
   Vigor.—Medium.
   Growth type.—Central leader, spindle.
   Leaves.—Length: 17.2 cm. Width: 8.5 cm. Form: Broad and obovate. Base: Acute. Apex: Apiculate (acute). Margin: Dentate with symmetrical teeth. Pubescence: Upper surface: None. Lower surface: Present on veins. Color: Young leaves: Upper surface: Yellow green. Lower surface: Light green. Mature leaves: Upper surface: Green. Lower surface: Light green. Venation: Type: Dichotomous. Color: Upper surface: Green. Lower surface: Light green.
   Flowers.—Blooming timing: Late. Pollination requirements: Necessary, Incompatibility Group II. Petals: Number: 5. Shape: Circular. Margin: Overlapping. Color (when opening and fully opened): Upper surface: White. Lower surface: White. Reproductive organs: Stamens: Average of 30-32 per flower. Anthers: Yellow in color. Pollen: Yellow in color and high in amount. Stigma: Green in color:
   Fruit.—Size: Diameter: 29.4 mm. Skin: Tendency to crack: Intermediate. Color: Dark red. Ground color: Red. Flesh: Aroma: Present. Color: Red. Texture: Firm. Eating quality: Very good. Seeds: Number per fruit: 1. Form: Circular. Color: Beige. Stem: Length: Long; Greater than 40.0 mm. Color: Green.
   Tree winter hardiness.—Good.
   Bud winter hardiness.—Good.
   Drought tolerance.—Unknown.
   Disease/pest resistance or susceptibility.—Unknown.

I claim:

1. A new and distinct variety of cherry tree, as is herein illustrated and described.
